(19) United States
(12) Patent Application Publication
(10) Pub. No.: US 2013/0302450 A1
(43) Pub. Date: Nov. 14, 2013

(54) HAIR GROWTH AND FOSTERING AGENT

(71) Applicant: Michio Kawano, Mima (JP)

(72) Inventor: Michio Kawano, Mima (JP)

(73) Assignee: Kawano Mericlone Co., Ltd., Tokushima (JP)

(21) Appl. No.: 13/981,195

(22) PCT Filed: Feb. 9, 2011
(86) PCT No.: PCT/JP2011/079341

§ 371 Date: Jul. 23, 2013

(30) Foreign Application Priority Data
Feb. 9, 2011 (JP) 2011-026187

(51) Int. Cl. A61K 8/97; A61Q 7/00

(52) U.S. Cl. CPC A61K 8/97; A61Q 7/00
USPC 424/725

(57) ABSTRACT

Provided is a hair growth agent/hair tonic capable of improving the hair generation and growth effects and achieving the anti-canities effects. Specifically, the hair growth agent/hair tonic comprises, as an active ingredient, the extract obtained from the whole part of a plant belonging to the genus Cymbidium of the family Orchidaceae, and also has the anti-canities effect. Great Flower Marie Laurencin is particularly effective as the plant belonging to the genus Cymbidium of the family Orchidaceae. The amount of the extract to be added is 0.1 to 30% by weight, desirably 1.0 to 20% by weight, and particularly preferably 5.0 to 15% by weight.

### TECHNICAL FIELD

The present invention relates to a hair growth and fostering agent having prominent hair growth and fostering effects and anti-canities effects without causing side effects in the living body.

### BACKGROUND ART

Conventionally, various studies and developments have been made on hair growth and fostering agents, and chemically synthesized substances containing components such as hormonal agents, blood flow-promoting agents, and cell-activating agents are provided. However, there have been problems that although the majority of these hair growth and fostering agents have excellent hair generation and growth and fostering effects, they also cause side effects. Further, such a hair growth and fostering agent that can be expected to have anti-canities effects has not yet been obtained.

With an aim to achieve hair growth and fostering effects while improving safety, the present inventor has proposed a hair growth and fostering agent prepared by extracting an active ingredient present in natural plants, instead of using chemically synthesized materials. That is, the present inventor has proposed a hair growth and fostering agent, comprising an extract of the pseudobulb of Great Flower Marie Laurencin, which is a plant belonging to the genus Cymbidium of the family Orchidaceae, as an active ingredient in Patent Literature 1 (Japanese Patent No. 3513475).

### CITATION LIST

### Patent Literature

Patent Literature 1: Japanese Patent No. 3513475

### SUMMARY OF INVENTION

### Technical Problem

The hair growth and fostering agent described in Patent Literature 1 was highly safe since it contained a natural plant component as an active ingredient, and also achieved dramatically improved hair growth and fostering effects compared to prior products. However, on average, it requires a period of approximately six months to obtain the hair generation and growth effects after initiation of use. Although the above time period has been shortened compared to before, there is a demand for improving the hair growth and fostering effects for an even more immediate effect. Further, if a hair growth and fostering agent having anti-canities effects, namely, a hair growth and fostering agent capable of turning gray hair back to black hair, becomes available, such a hair growth and fostering agent is desirable for users.

In view of the foregoing, an object of the present invention is to provide a hair growth and fostering agent that is not only capable of further improving the hair growth and fostering effects, but also effective against canities.

### Solution to Problem

The inventor of the present application strove to achieve the aforementioned object. As a result, he has found that an extract obtained from the whole part, not from only the pseudobulb, of a plant belonging to the genus Cymbidium of the family Orchidaceae has excellent hair growth and fostering effects as well as anti-canities effects with immediate actions, thereby leading to the completion of the present invention.

Plants belonging to the genus Cymbidium are monocotyledons of the family Orchidaceae, which are distributed and grow naturally mainly in the temperate and tropical regions of Asia. The plants have peculiar pseudobulbs, and there are approximately 70 species of Cymbidium plants. Further, breeding has been undertaken in plants belonging to the genus Cymbidium, and a large number of breeds are now registered under the Plant Variety Protection and Seed Act of Japan. As a representative variety of plants belonging to the genus Cymbidium, including native species, cym. goeringii, cym. kanran, cym. sinense, cym. ensifolium, cym. pumilum, Cym. Great Flower “Marie Laurencin”, Cym Rose Wine “Shinseiki”, Cym. Great Flower “Ballerina”, Cym. Lucky Flower “Anmitsuhime”, Cym. Great Katy “Mariko”, Cym. Lucky Gloria “Aguri”, Cym. Lucky Gloria “Fukunokami”, Seaside “Saya”, Cym. Satin Doll “Golden Yellow”, Cym. Great Katy “Little Laurencin”, Angelico “Fukumusume”, and the like are known. Among native species, some are used as medicines or foods in regions such as China, Southeast Asia, and Australia.

As described above, there are an extremely large number of plants belonging to the genus Cymbidium of the family Orchidaceae. Although no limitation is imposed, among those plants, a variety registered under the name of Great Flower Marie Laurencin (Variety Registration No. 2841) under the Plant Variety Protection and Seed Act of Japan is preferable since it has particularly excellent hair growth and fostering effects as well as anti-canities effects with immediate actions.

The extract of a plant belonging to the genus Cymbidium of the family Orchidaceae may be an extract obtained by using the whole part of a plant belonging to the genus Cymbidium of the family Orchidaceae as the extraction raw material. The whole part of a plant refers to all the parts of a plant combining the aerial and underground parts such as the flowers, leaves, flower stalks, pseudobulbs, and roots. It is preferable to use the whole part of a plant belonging to the genus Cymbidium as the extraction raw material at the flowering time, especially at the peak flowering time; however, the timing may not be limited to the flowering time.

For the adjustment of the extract of the whole part of a plant belonging to the genus Cymbidium of the family Orchidaceae, any of the physical extraction method, in which the whole part of a plant belonging to the genus Cymbidium of the family Orchidaceae is compressed directly or after being shredded into pieces of appropriate size or pulverized by a grinder, the solvent extraction method, and the vacuum distillation method can be adopted, and extracts thereby obtained with various kinds of solvents, their dilutions, concentrates, or dried powders are used as the extract. Examples of the extraction solvent include a hydrophilic solvent such as water, methanol, ethanol, propylene glycol, 1,3-butylene glycol, glycerin, and acetone, and a mixture of two or more kinds thereof may also be used. Further, the extract to be prepared may be either a liquid or a solid.

For example, when ethanol extraction is employed, a method for producing the extract of the whole part of a plant belonging to the genus Cymbidium of the family Orchidaceae is adopted such that the whole part of a plant belonging to the genus Cymbidium of the family Orchidaceae is immersed directly or after being shredded into pieces of appropriate size or pulverized by a grinder in a 50% aqueous solution of ethanol at a weight ratio of 2:7 (whole plant:50% aqueous solution of ethanol) and extraction operation is carried out with occasional stirring, and the residue is filtered off. Further, when hot water extraction is employed, the extract of the whole part of a plant belonging to the genus Cymbidium of the family Orchidaceae can be produced by adding 100 ml of purified water to 5 g of the pulverized plant obtained similarly to the above, followed by thermal extraction in a boiling water bath for approximately two hours, and then adding an equivalent amount of 95% ethanol, and after leaving the resulting mixture to stand, filtering off the residue. It should be noted that the extraction method is not limited to those described above.

The extracts described above may be used in the hair growth and fostering agent directly or after arbitrarily adjusting by, for example, drying, concentrating, diluting, or mixing with other components according to the application form.

Also, the amount of the extract to be added to the hair growth and fostering agent ranges preferably from 0.1 to 30% by weight, desirably from 1.0 to 20% by weight. When the content of the extract was less than 0.1% by weight or more than 30% by weight, in either case, preferable hair growth, hair fostering and anti-canities effects could not be obtained. The most preferable added amount is 5.0 to 15% by weight, at which amount the best hair growth, hair fostering, and anti-canities effects were exhibited.

As described above, pharmaceutical products, quasi drugs, or cosmetics can be prepared by blending the extract obtained from the whole part of a plant belonging to the genus Cymbidium of the family Orchidaceae with an aim to obtain such functional effects as hair growth, hair fostering, and anti-canities effects.

As the dosage form of the hair growth and fostering agent of the present invention, an externally applied agent is preferable, and it is preferably provided in the form of embrocations such as ointments, lotions, tonics, sprays, suspensions, and emulsions. As more specific usage, for example, the hair growth and fostering agent of the present invention can be used in various forms such as hair revitalizing agents, hair tonics, hair lotions, hair creams, shampoos, hair rinses, hair treatments, and hair colors.

To the hair growth and fostering agent of the present invention, moisturizing agents, surfactants, dyes, fragrances, enzymes, hormones, vitamins, ultraviolet ray absorbers, ultraviolet ray shielding agents, solvents, stabilizers, plasticizers, lubricants, solubilizers, reducing agents, buffers, sweeteners, bases, volatilization aids, adsorbents, taste masking agents, synergists, binders, suspending agents, antioxidants, gloss agents, coating agents, humectants, cooling agents, softeners, emulsifiers, excipients, antiseptic agents, preservatives, and the like can be added according to an ordinary method so long as the effect of the present invention is not impaired.

### Advantageous Effects of Invention

The present invention can provide a hair growth and fostering agent having hair generation, hair growth, and anti-canities effects with excellent immediate actions by including the extract obtained from the whole part of a plant belonging to the genus Cymbidium of the family Orchidaceae as an active ingredient.

### BRIEF DESCRIPTION OF DRAWINGS

FIG. 1 is a photograph showing the condition in the course of usage in a test user A in Study Example 2.

FIG. 2 is a photograph showing the condition approximately 10 months after FIG. 1.

FIG. 3 is a photograph showing the condition of a test user B at the beginning of use in Study Example 2.

FIG. 4 is a photograph showing the condition approximately 1 month after FIG. 3.

FIG. 5 is a photograph showing the condition of a test user C at the beginning of use in Study Example 2.

FIG. 6 is a photograph showing the condition approximately 4 months after FIG. 5.

FIG. 7 is a photograph showing the condition of a test user D at the beginning of use in Study Example 2.

FIG. 8 is a photograph showing the condition approximately 6 months after FIG. 7.

FIG. 9 is a photograph showing the condition of a test user E at the beginning of use in Study Example 2.

FIG. 10 is a photograph showing the condition approximately 1 month after FIG. 9.

### DESCRIPTION OF EMBODIMENTS

The present inventor conducted intensive studies on plant extracts having the hair growth and fostering effects. As a result, he has found that excellent hair growth, hair fostering, and anti-canities effects can be obtained when the extract obtained from the whole part of a plant belonging to the genus Cymbidium of the family Orchidaceae, but not the extract of the pseudobulb of a plant belonging to the genus Cymbidium of the family Orchidaceae, is contained. The present hair growth and fostering agent has achieved the best immediate action ever, and was confirmed by a variety of tests to exert high hair growth, hair fostering, and anti-canities effects in a period of as short as approximately one month. Therefore, the extract obtained from the whole part of a plant belonging to the genus Cymbidium of the family Orchidaceae can be used as an active ingredient of a hair growth and fostering agent and an anti-canities agent.

### EXAMPLES

### Preparation of the Extract of the Whole Part of a Plant Belonging to the Genus Cymbidium of the Family Orchidaceae

The whole plant of blooming Great Flower Marie Laurencin (Variety Registration No. 2841), which is a plant belonging to the genus Cymbidium, is shredded. The shredded plant is then added to and immersed in a 50% aqueous solution of ethanol at a weight ratio of 2:7 (whole plant:50% aqueous solution of ethanol) to carry out extraction for one month at room temperature. Subsequently, the residue was filtered off and the resulting ethanol extract was used as the extract of the whole part of a plant belonging to the genus Cymbidium of the family Orchidaceae.

### Formulation Example

### Hair tonic

This Formulation Example is a formulation example of a hair tonic, and its composition is as shown below.

| Formulation component | % by weight |
|---|---|
| The aforementioned extract of the whole | 10.5% by weight |
| part of a plant belonging to the genus | |
| Cymbidium of the family Orchidaceae | |
| Ethanol | 47.05% by weight |
| Cooling agent (1-menthol) | 0.30% by weight |
| Antiseptic agent (Photosensitizer 301) | 0.002% by weight |
| Ginger root extract | 0.10% by weight |
| Humectant | 2.652% by weight |
| Common water | 10.0% by weight |
| Purified water | Balance |

Also, examples of the humectant include, but are not limited to, sugar having moisturizing actions such as maltitol, a Rehmannia Chinensis root extract, a Tilia Cordata flower extract, a seaweed extract, and a liquid rice extract hydrolysate. These components can be blended singly or in combination. Humectants have moisturizing actions, and thus can impart appropriate moisture to the scalp and hair.

### Comparative Example

### Hair Tonic

First of all, the pseudobulb of blooming Great Flower Marie Laurencin (Variety Registration No. 2841), which is a plant belonging to the genus Cymbidium, is shredded. The shredded pseudobulb is then added to and immersed in a 50% aqueous solution of ethanol at a weight ratio of 2:7 (whole plant:50% aqueous solution of ethanol) to carry out extraction for one month at room temperature. Subsequently, the residue was filtered off and the resulting ethanol extract was used as the extract of the pseudobulb of a plant belonging to the genus Cymbidium of the family Orchidaceae. Using the extract thus obtained, a hair tonic of Comparative Example was formulated. The composition of the hair tonic of Comparative Example only differs from that of the hair tonic of Formulation Example shown above in that the “extract of the whole part of a plant belonging to the genus Cymbidium of the family Orchidaceae, 10.5% by weight” is replaced by the “extract of the pseudobulb of a plant belonging to the genus Cymbidium of the family Orchidaceae, 10.5% by weight”, while the rest of the composition remains the same. In light of this, explanation is omitted.

Hereinbelow, the following studies were conducted in order to confirm the effect of each of Formulation Example (extract of whole plant) and Comparative Example (extract of only pseudobulb).

### Study Example 1

Twenty seven males and females were assigned to subjects and used the hair tonic prepared according to Formulation Example, and their feedback is summarized in Table 1 below. Also, 20 males and females were assigned to subjects and used the hair tonic prepared according to Comparative Example, and their feedback is summarized in Table 2. As to the study method, the subjects were instructed to apply and spread the aforementioned hair tonic on the head in a normal usage mode and thoroughly massage the head, and perform the above operation once or twice a day.

TABLE 1

| | | | After use | | |
|---|---|---|---|---|---|
| | | | Time between | | |
| | | | initiation of | | |
| | | | use and | | |
| | | | manifestation | Hair growth and | |
| No. | Sex and Age | Before use | of effects | fostering effects | Anti-canities effects |
| Test | Male, 67 | Almost entirely | Approx. 1 | Hair became thick and | Hair on the back of the |
| user 1 | | gray head of | month | vitalized. | head started to turn |
| | | fine, fragile | | | back to black. After |
| | | hair | | | considerable |
| | | | | | improvement, hair |
| | | | | | generally turned back |
| | | | | | to black. |
| Test | Male, 57 | Bald on top | 2 to 3 months | Hair became so thick | |
| user 2 | | | after initiation | and vitalized overall | |
| | | | of use | that it was mistaken for | |
| | | | | a wig. Downy hair | |
| | | | | started to and continued | |
| | | | | to regrow on the once- | |
| | | | | bald top of the head. | |
| Test | Male, 64 | Generally thin | 1 to 2 months | Hair thinning alleviated | Back of the head |
| user 3 | | | | overall and hair became | started to turn back to |
| | | | | vitalized and thickened. | black. |
| Test | Male, 64 | Bald on top | 1 to 2 months | Hair started to regrow | |
| user 4 | | | | on top of the head, | |
| | | | | followed by rapid | |
| | | | | regrowth. | |
| Test | Male, 46 | | | Hair texture rapidly | |
| user 5 | | | | improved with | |
| | | | | noticeable hair | |
| | | | | regrowth in the front of | |
| | | | | the head. Hair was | |
| | | | | volumized overall. | |
| Test | Male, 63 | | 1 to 2 months | Hair became thick and | Canities alleviated and |
| user 6 | | | | vitalized with no more | started to turn back to |
| | | | | hair loss. People | black. |
| | | | | noticed the restoration | |
| | | | | of hair. | |
| Test | Male, 63 | | 1 to 2 months | Hair became thick and | Hair on temples |
| user 7 | | | | vitalized and volumized | considerably improved. |
| | | | | overall. | |
| Test | Male, 42 | Becoming | 1 to 2 months | Hair improves while | A fist-sized chunk of |
| user 8 | | noticeably thin | | rapidly becoming thick | gray hair was |
| | | | | and vitalized. | noticeable in the front |
| | | | | | of the head but people |
| | | | | | pointed out that it |
| | | | | | became unnoticeable. |
| Test | Male, 64 | Hair was | 1 to 2 months | Hair was rapidly | People commented that |
| user 9 | | thinning | | improving, relieving | gray hair was generally |
| | | generally. | | him from concern. | turning back to black. |
| | | Particularly | | | |
| | | concerned | | | |
| | | about partial | | | |
| | | baldness on the | | | |
| | | side of the head | | | |
| Test | Male, 63 | | 1 to 2 months | Hair became resilient | Gray hair turned back |
| user 10 | | | | and thick and | to black. |
| | | | | volumized overall. | |
| Test | Male, 63 | Hair was | 1 to 2 months | Hair became resilient | Canities was also being |
| user 11 | | thinning | | and thick and coarse to | alleviated. |
| | | generally. | | touch. | |
| Test | Female, 56 | | Approx. 2 | Hair loss was reduced | |
| user 12 | | | months | while hair became | |
| | | | | resilient and shiny. | |
| | | | | Hairstyling can be done | |
| | | | | with a hair dryer and | |
| | | | | hands, relieving her | |
| | | | | from hair-related | |
| | | | | concerns. | |
| Test | Female, 37 | Had lost hair | 1 month | Hair became strong and | Graying hair was |
| user 13 | | from the | | hair loss was also | turning back to black. |
| | | forehead to the | | visibly reduced. | |
| | | top of the head | | | |
| | | due to | | | |
| | | radiotherapy | | | |
| Test | Female, 58 | Concerned | 1 month | Hair loss was reduced. | |
| user 14 | | about hair | | She also likes slick feel | |
| | | falling out | | to fingers after use. | |
| | | during | | | |
| | | shampooing | | | |
| | | and brushing | | | |
| Test | Female, 51 | Feels the | | Had her hair cut | |
| user 15 | | weight and | | approx. 1 month after | |
| | | growth of hair | | use, and she started to | |
| | | approx. 1.5 to 2 | | feel the weight and | |
| | | months after | | growth of hair approx. | |
| | | haircut | | 1 week after haircut. | |
| Test | Female, 57 | | A few days | Hair loss was | |
| user 16 | | | | considerably reduced in | |
| | | | | a few days after use. | |
| | | | | Combing became easy | |
| | | | | and hair was | |
| | | | | volumized, seemingly | |
| | | | | vitalized. Although | |
| | | | | having sensitive skin, | |
| | | | | the product does not | |
| | | | | cause rush and is mild | |
| | | | | to the skin. | |
| Test | Female, 52 | | 50 days | Hair loss was reduced | |
| user 17 | | | | and hair became shinier | |
| | | | | than before. She was | |
| | | | | once annoyed with | |
| | | | | itchiness but is less | |
| | | | | annoyed now. | |
| Test | Female, 47 | Flat due to fine | | Gained light, airy hair | |
| user 18 | | hair | | with reduced hair loss. | |
| | | | | Family commented that | |
| | | | | her hair was volumized. | |
| Test | Female, 51 | | 1 month | The product was mild | |
| user 19 | | | | to the scalp and hair | |
| | | | | became moistened and | |
| | | | | shiny with reduced hair | |
| | | | | loss. | |
| Test | Female, 57 | Hair on top of | 1 month | Hair is gradually | |
| user 20 | | the head is fine | | becoming resilient. | |
| | | and lacks | | | |
| | | resilience. | | | |
| Test | Female, 58 | | 40 days | Dandruff and itchiness | |
| user 21 | | | | are gone and hair | |
| | | | | became resilient and | |
| | | | | volumized. Besides | |
| | | | | that, hair grew faster. | |
| Test | Female, 53 | Concerned | 1 month | Hair became resilient | |
| user 22 | | about limp hair | | and firm. | |
| Test | Female, 58 | | 1.5 months | Relieved from concern | |
| user 23 | | | | about hair loss, and | |
| | | | | itchiness was also | |
| | | | | reduced. | |
| Test | Female, 60 | | 2 months | | Becoming less |
| user 24 | | | | | concerned about gray |
| | | | | | hair on the side of the |
| | | | | | head, which used to |
| | | | | | annoy her. |
| Test | Female, 38 | | 2 months | Hair gained an | Once gray hair was |
| user 25 | | | | improved touch. | turning to brown, |
| | | | | | making gray hair less |
| | | | | | noticeable. |
| Test | Female, 61 | Concerned | 2.5 months | People commented that | Becoming less |
| user 26 | | about thinning | | hair was volumized. | concerned about gray |
| | | on top of the | | | hair on the hair line. |
| | | head | | | |
| Test | Female, 34 | | 1.5 months | Hair became shiny. | Gray hair was also |
| user 27 | | | | | reduced. |

TABLE 2

| | | | After use | | |
|---|---|---|---|---|---|
| | | | Time between | | |
| | | | initiation of | | |
| | | | use and | | |
| | | | manifestation | Hair growth and | |
| No. | Sex and Age | Before use | of effects | fostering effects | Anti-canities effects |
| Test | Male, 57 | | Half a year | Hair on the front part seems to be | |
| user 28 | | | | increasing. | |
| Test | Male, 60 | | Half a year | Hair on top of the head started to | |
| user 29 | | | | grow in thickness. | |
| Test | Male, 63 | | Half a year | Downy hair gradually regrew on | |
| user 30 | | | | the back of the head. | |
| Test | Male, 64 | | Half a year | The product is non-sticky and | |
| user 31 | | | | feels fresh, and is also effective. | |
| Test | Male, 64 | | Half a year | Hair seems to become firm. | |
| user 32 | | | | Once short hair grew longer and | |
| | | | | seems to be increasing. | |
| Test | Male, 67 | | Half a year | Hair remains dandruff-free and | |
| user 33 | | | | non-itchy, and hair started to | |
| | | | | regrow in once-hairless areas. | |
| Test | Male, 87 | Bald on top | 7, 8 months | Short downy hair started to | |
| user 34 | | | | regrow in the back of the head, | |
| | | | | which is now 4 cm long. | |
| Test | Male | | Half a year | Downy hair slightly increased on | |
| user 35 | | | | the hairline, and hair was | |
| | | | | volumized overall. | |
| Test | Male, 60 | | 5, 6 months | Hair loss was reduced. | |
| user 36 | | | | | |
| Test | Male, 68 | | Half a year | Downy hair is growing on the | |
| user 37 | | | | once completely bald head. | |
| Test | Female, 70 | Thin hair | 7 months | Once limp hair was volumized, | |
| user 38 | | | | enabling her to go out without a | |
| | | | | hat. | |
| Test | Female | Fine, thin | Half a year | Hair became firm with reduced | |
| user 39 | | hair | | hair loss. | |
| Test | Female, 78 | | Half a year | Downy hair started to regrow on | Gray hair almost |
| user 40 | | | | the sides of the forehead, where | disappeared. |
| | | | | hair was thinning. | |
| Test | Female, 84 | | Half a year | | Hair turned back |
| user 41 | | | | | to black. |
| Test | Female, 73 | | Half a year | Hair started to increase. | |
| user 42 | | | | | |
| Test | Female, 70 | | 7 months | Became less concerned about thin | |
| user 43 | | | | hair on top of the head. | |
| Test | Female, 77 | | 7 months | Short hair started to regrow. | |
| user 44 | | | | | |
| Test | Female, 73 | | Half a year | Felt the product was effective. | |
| user 46 | | | | | |
| Test | Female, 60 | | Half a year | Hairdresser noticed improved hair | |
| user 47 | | | | texture and downy hair started to | |
| | | | | regrow on the front of the head. | |
| Test | Female, 81 | | Half a year | Hair started to gradually increase. | |
| user 48 | | | | | |

As is apparent from the above results, the time period until the test users felt any sort of effect was as short as 1 to 2 months with Formulation Example containing the extract of the whole part of a plant belonging to the genus Cymbidium of the family Orchidaceae. This was much quicker compared to before, and almost all the test users obtained the hair growth and fostering effects. The newly grown hair was not limited to downy hair and thick hair also started to regrow. Hair gained resilience and firmness and hair loss was also reduced.

It was further found that many test users also felt profound anti-canities effects with Formulation Example containing the extract of the whole part of a plant belonging to the genus Cymbidium of the family Orchidaceae. With regard to this anti-canities effect, although the mechanisms are unknown, many test users commented that it was such that existing hair turned back to black gradually from the root, rather than newly growing hair turning to black gradually from the root, revealing that canities were alleviated in a short period of time.

In contrast, the test monitors obtained the hair generation and growth effects with Comparative Example containing the extract of the pseudobulb of a plant belonging to the genus Cymbidium of the family Orchidaceae only after approximately half a year of use. Moreover, remarkable effects have not been obtained with Comparative Example with only two test users experiencing the anti-canities effects.

As described above, it was confirmed that the hair growth and fostering agent containing the extract of the whole part of a plant belonging to the genus Cymbidium of the family Orchidaceae according to the present invention was effective in terms of both the hair growth and fostering effects and anti-canities effects.

### Study Example 2

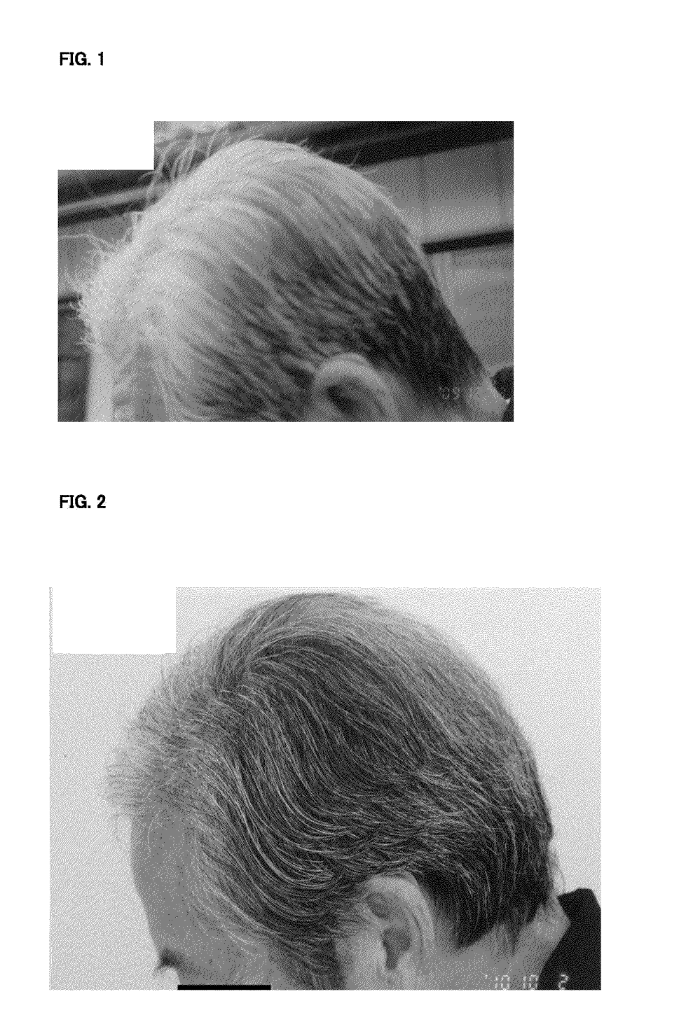

Subsequently, five males ranging from their 40's to 70's were assigned to subjects. The changes in their hair resulting from the use of the hair tonic adjusted according to the aforementioned Formulation Example were recorded photographically and shown in FIGS. 1 to 10. As to the study method, the subjects were instructed to apply and spread the aforementioned hair tonic on the head in a normal usage mode and thoroughly massage the head, and perform the above operation once or twice a day.

The test user A started to use the hair tonic of Formulation Example on Oct. 25, 2009. Thereafter, he noticed that the back of the head was turning back to black, and had his hair photographed on Dec. 13, 2009, which was 48 days after initiation of use. The photograph is shown in FIG. 1. That is, he experienced the anti-canities effect in a duration of use of approximately 1.5 months. Approximately 10 months later, another photograph was taken on Oct. 2, 2010, which is shown in FIG. 2. Comparing FIG. 1 with FIG. 2, it is found that the color of the hair generally turned back to black with continuous use, showing a continuous anti-canities effect.

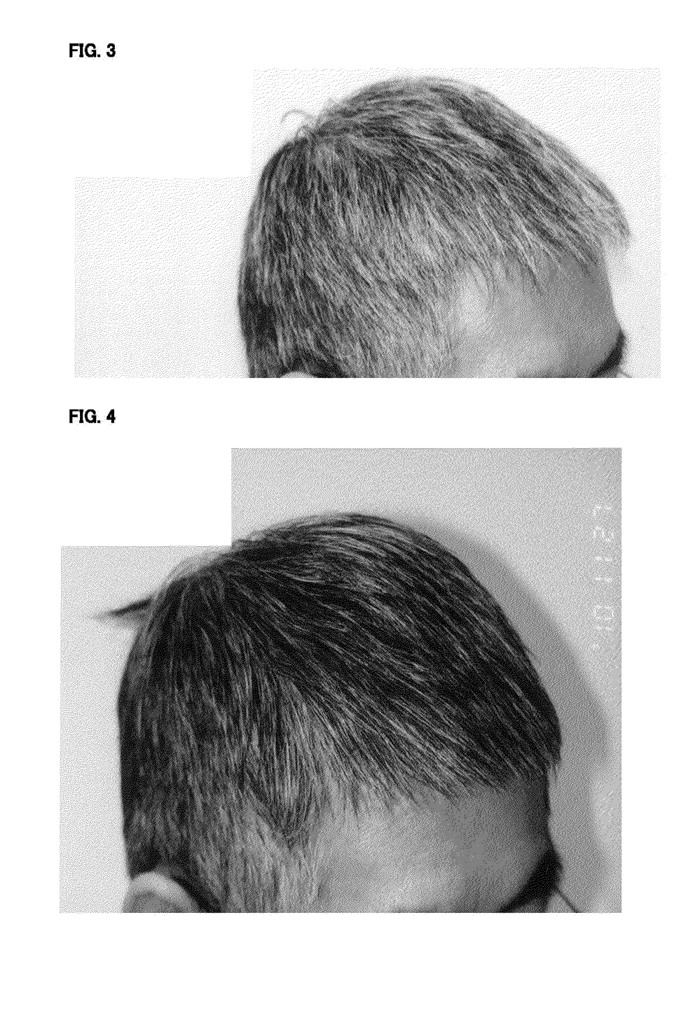

The test user B had more gray hair than black hair according to the condition as shown in the photograph of FIG. 3, which was taken at the beginning of use on Oct. 25, 2010. However, according to the condition as shown in the photograph of FIG. 4, which was taken approximately 1 month later on Nov. 27, 2010, gray hair almost completely disappeared, revealing that canities were alleviated in as short as one month. Moreover, hair has gained resilience and firmness.

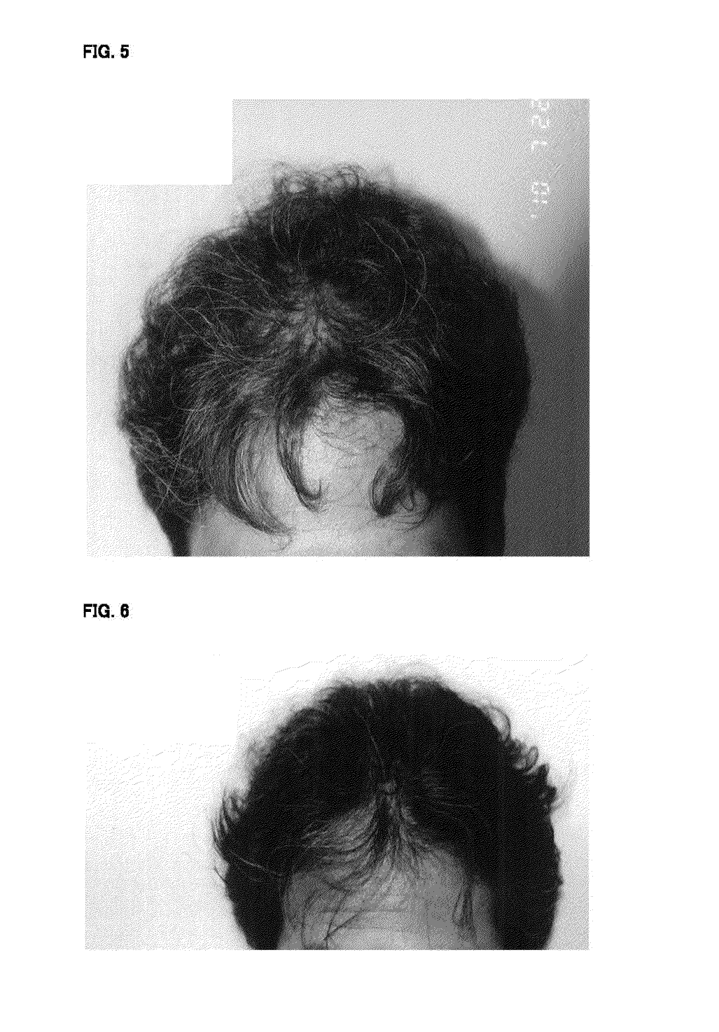

Compared to the condition as shown in the photograph of FIG. 5, which was taken at the beginning of use on Jul. 22, 2010, the test user C achieved hair growth, hair fostering, and anti-canities effects with the use of the hair tonic of Formulation Example. As of Nov. 15, 2010, which was approximately four months later, the anti-canities effects and hair growth and fostering effects were prominently exhibited as shown in the photograph of FIG. 6.

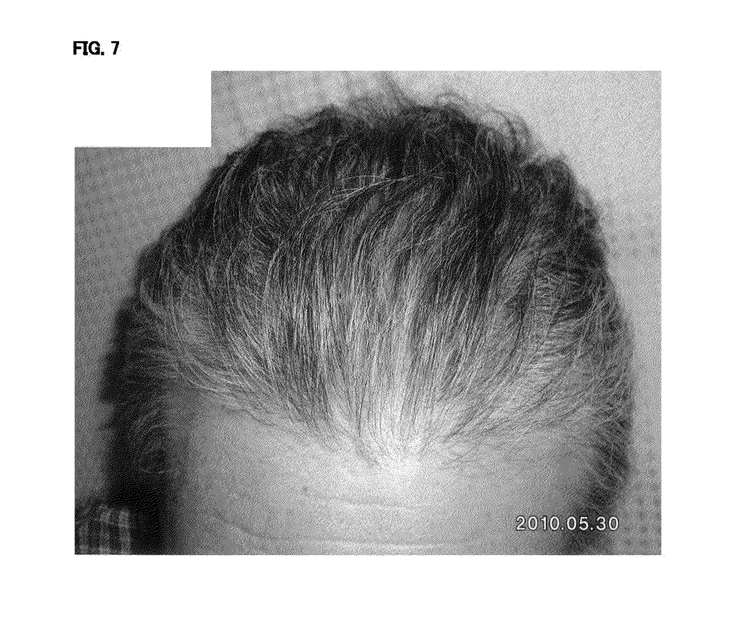

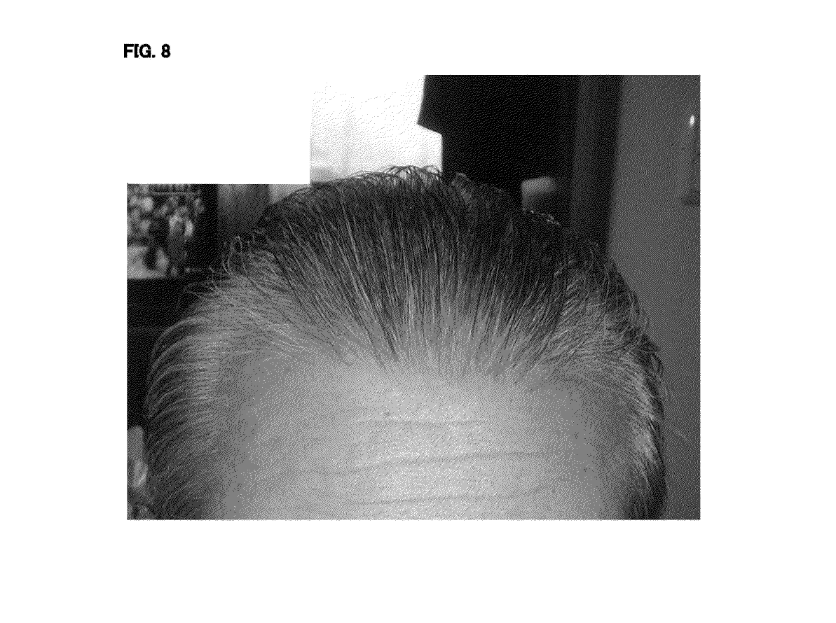

Despite the fact that the test user D is relatively elderly (73 years old), compared to the condition as shown in the photograph of FIG. 7, which was taken at the beginning of use on May 30, 2010, canities has been alleviated particularly on top of the head, and moreover, hair has increased according to the condition as shown in the photograph of FIG. 8, which was taken approximately six months later on Nov. 23, 2010. That is, the hair generation, hair growth, and anti-canities effects were obtained even in a relatively elderly individual, in whom the revitalization of hair cannot be expected very much.

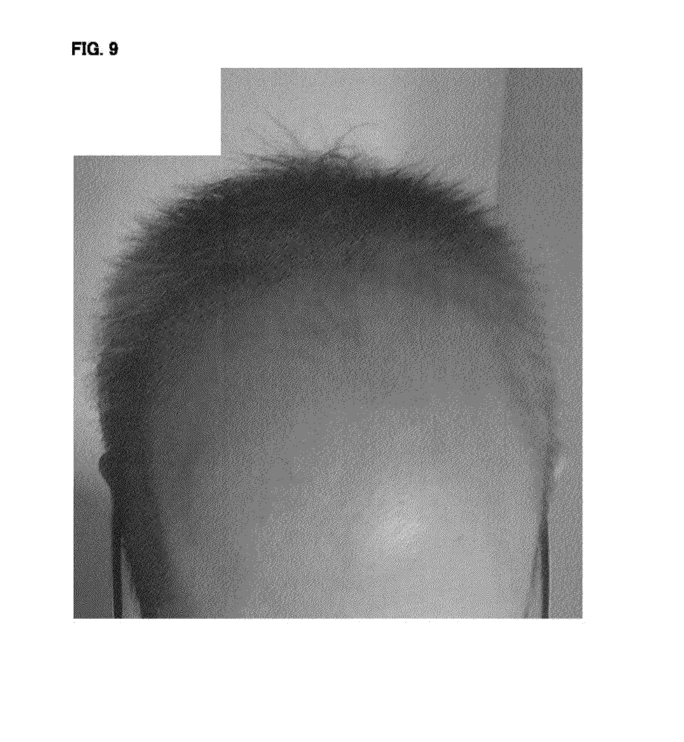

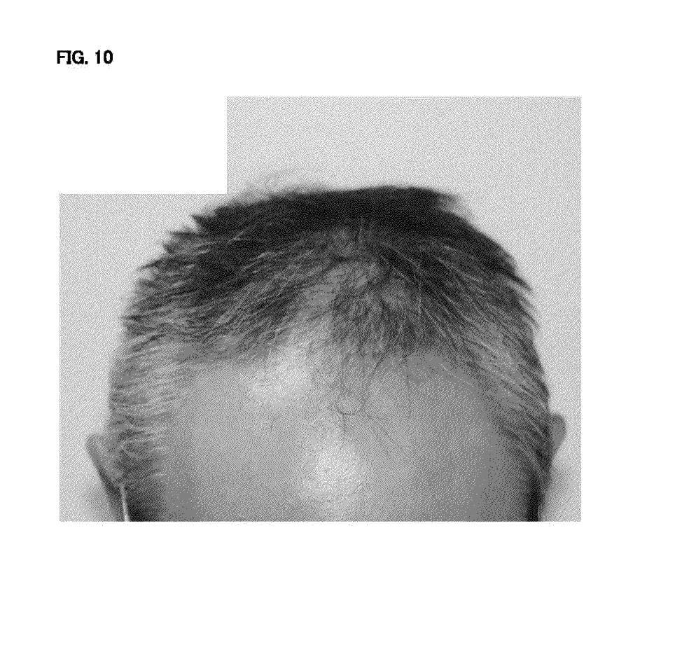

In the test user E, it is found that, compared to the condition as shown in the photograph of FIG. 9, which was taken at the beginning of use on Oct. 7, 2010, canities has been alleviated while new hair has grown, and moreover, hair has increased according to the condition of the photograph of FIG. 10, which was taken only approximately one month later on Nov. 15, 2010.

As described above, the hair growth and fostering agent of the present invention successfully produced the hair growth and fostering effects and anti-canities effects in a short period of time.

### INDUSTRIAL APPLICABILITY

The present invention can be usefully employed as a hair growth and fostering agent—having prominent hair growth and fostering effects as well as anti-canities effects without causing side effects in the living body.

1-6. (canceled)

7. A hair growth and fostering agent having an anti-canities effect, comprising, as an active ingredient, an extract obtained from a whole part of Great Flower Marie Laurencin, which is a plant belonging to a genus Cymbidium of the family Orchidaceae.

8. The hair growth and fostering agent according to claim 7, wherein the hair growth and fostering agent comprises the extract in an amount of 0.1 to 30% by weight.

9. The hair growth and fostering agent according to claim 7, wherein the hair growth and fostering agent comprises the extract in an amount of 1.0 to 20% by weight.

10. The hair growth and fostering agent according to claim 7, wherein the hair growth and fostering agent comprises the extract in an amount of 5.0 to 15% by weight.
